(19) United States
(12) Patent Application Publication
(10) Pub. No.: US 2009/0227579 A1
(43) Pub. Date: Sep. 10, 2009

(54) Pteridine Derivatives and their Use as Cathespin Inhibitors

(75) Inventors: Robert Andrew Heald, Essex (GB); Andrew David Morley, Leicestershire (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/305,404

(22) PCT Filed: Jun. 20, 2007
(86) PCT No.: PCT/GB07/02269

§ 371 Date: Dec. 18, 2008

Related U.S. Application Data
(60) Provisional application No. 60/816,157, filed on Jun. 23, 2006.

(51) Int. Cl. A61K 31/5377; C07D 475/06; A61K 31/4985; A61P 19/02; A61P 19/10; A61P 35/00; A61P 35/04; C07D 401/14; C07D 413/14

(52) U.S. Cl. 514/234.2; 544/258; 514/249; 544/118

(57) ABSTRACT

The present invention relates to compounds and compositions for treating diseases associated with cysteine protease activity. The compounds are reversible inhibitors of cysteine proteases S, K, F, L and B. Of particular interest are diseases associated with Cathepsin K.

The present invention relates to compounds and compositions for treating diseases associated with cysteine protease activity. The compounds are reversible inhibitors of cysteine proteases S, K, F, L and B. Of particular interest are diseases associated with Cathepsin S. In addition this invention also discloses processes for the preparation of such inhibitors.

### BACKGROUND OF THE INVENTION

Cathepsin K is a member of the papain superfamily of cysteine proteases which also encompasses Cathepsins B, H, L, O and S. Cathepsin K plays a key role in the processing of invariant chain in MHC class II complexes allowing the complex to associate with antigenic peptides. MHC class II complexes are then transported to the surface of the cell for presentation to effector cells such as T cells. The process of antigen presentation is a fundamental step in initiation of the immune response. In this respect inhibitors of cathepsin K could be useful agents in the treatment of inflammation and immune disorders such as, but not limited to, asthma, rheumatoid arthritis, multiple sclerosis and Crohn's disease. Cathepsin K has also been implicated in a variety of other diseases involving extracellular proteolysis such as the development of emphysema in COPD through degradation of elastin and in Alzheimers disease.

Other Cathepsins notably L have been shown to degrade bone collagen and other bone matrix proteins. Inhibitors of these cysteine proteases would be expected to be useful in the treatment of diseases involving bone resorption such as osteoporosis.

The present invention therefore provides a compound of formula (I)

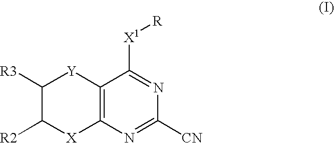

in which:

### X is NR¹ or O;

### Y is O or NR⁴;

X¹ is a bond, NH or Nalkyl,
R is a 4, 5, 6 or 7-membered saturated monocyclic or bicyclic ring optionally containing one or more O, S(O)n or N atoms which can be optionally substituted by alkyl, C₃₋₆ cycloalkyl, or a spirocyclic group comprising 3-5 membered rings or (CH₂)nX where X is amino, hydroxy, OR⁴, cyano, trifluoromethyl, carboxy, CONR⁵R⁶, SO₂NR⁵R⁶, SO₂R⁴, NR⁴SO₂R⁴, NR⁴COR⁴, C₁₋₆ alkyl, C₁₋₆ alkoxy, SR⁴ or NR⁵R⁶ where R4 is hydrogen, C₁₋₆ alkyl or C₃₋₆ cycloalkyl, R⁵ and R⁶ are independently hydrogen, C₁₋₆ alkyl, or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶;
or R is a group —(CH₂)nY(CH₂)pR⁷ where n and p are independently 0, 1 or 2 and Y is a bond, O, S(O)n or NR⁸ where R⁸ is hydrogen, C₁₋₆ alkyl or C₃₋₆ cycloalkyl)
R¹ is hydrogen, C₁₋₆ alkyl or C₃₋₇ cycloalkyl, both of which can be optionally substituted by alkyl (including branching), cycloalkyl (useful to include this a spiocyclo as well), or (CH2)nX where X=amino, hydroxy, OR4, cyano, trifluoromethyl, carboxy, CONR⁵R⁶, SO₂NR⁵R⁶, SO₂R⁴, NR⁴SO₂R⁴, NR⁴COR⁴, C₁₋₆ alkyl, C₁₋₆ alkoxy, SR⁴ or NR⁵R⁶; or
R¹ is a group —(CH₂)nY(CH₂)pR⁷ where n and p are independently 0, 1 or 2 and Y is a bond, O, S(O)n or NR⁸ where R⁸ is hydrogen, C₁₋₆ alkyl or C₃₋₆ cycloalkyl;
R² is hydrogen or C₁₋₆ alkyl;
R³ is hydrogen or C₁₋₆ alkyl;
R⁴ is hydrogen, C₁₋₆ alkyl or C₃₋₆ cycloalkyl, and
R⁵ and R⁶ are independently hydrogen, C₁₋₆ alkyl;
R⁷ is a 3- to 7-membered saturated ring optionally containing one or more O, S or N atoms (sulphur may be in the form S(O)n), or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶;
or R⁷ is hydrogen, amino, hydroxy, OR⁴, cyano, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶,
and pharmaceutically acceptable salts or solvates thereof.

In the context of the present specification, unless otherwise indicated, an alkyl or alkenyl group or an alkyl or alkenyl moiety in a substituent group may be linear or branched. Aryl groups include phenyl and naphthyl.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

In one embodiment of the invention X is NR¹.

In one embodiment of the invention Y is NH or O, more preferably Y is NH.

In one embodiment of the invention X¹ is a bond, NH, NMe,

Suitably R is a 5- or 6-membered saturated ring containing one or more O, S or N atoms, or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶;

In one embodiment of the invention R is a 5-7-membered saturated ring containing one or more O, S or N atoms, more preferably R is cyclopropyl, cyclohexyl, morpholine or piperidine. Most preferably R is morpholine. Preferred substituents include halogen.

Suitably R¹ is hydrogen, C₁₋₆ alkyl or C₃₋₆ cycloalkyl, both of which can be optionally substituted by hydroxyl or amino, or

R¹ is a group —(CH₂)nY(CH₂)pR⁷ where n and p are independently 0, 1 or 2 and Y is a bond, O or NR⁸ where R⁸ is hydrogen, C₁₋₆ alkyl or C₃₋₆ cycloalkyl;
and R⁷ is a 5- or 6-membered saturated ring containing one or more O, S or N atoms, or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶ where R⁴ is hydrogen, C₁₋₆ alkyl or C₃₋₆ cycloalkyl, R⁵ and R⁶ are independently hydrogen, C₁₋₆ alkyl;

In one embodiment of the invention R¹ hydrogen, C₁₋₆ alkyl substituted by hydroxyl or amino, C₃₋₆ cycloalkyl, phenyl optionally substituted by halogen or R¹ is a group —(CH₂)nO(CH₂)pR⁷ where n is 2 and p is 1 and R⁷ is phenyl. In one preferred embodiment of the invention R¹ is hydrogen, CH₂CH₂OH, CH₂CH₂NH₂, cyclopentyl, t-butyl, CH₂CH₂OCH₂Ph, phenyl or chlorophenyl.

In one embodiment of the invention R² and R³ are both hydrogen.

Preferred compounds of the invention include:
8-(2-Benzyloxy-ethyl)-4-(cyclopentyl-methyl-amino)-5,6,7,8-tetrahydro-pteridine-2-carbonitrile
8-(2-Benzyloxy-ethyl)-4-piperidin-1-yl-5,6,7,8-tetrahydro-pteridine-2-carbonitrile
8-Cyclopentyl-4-morpholin-4-yl-5,6,7,8-tetrahydro-pteridine-2-carbonitrile
4-Morpholin-4-yl-8-phenyl-5,6,7,8-tetrahydro-pteridine-2-carbonitrile
8-(2,2-Dimethyl-propyl)-4-morpholin-4-yl-5,6,7,8-tetrahydro-pteridine-2-carbonitrile
8-(2-Hydroxy-ethyl)-4-piperidin-1-yl-5,6,7,8-tetrahydro-pteridine-2-carbonitrile
4-(4,4-Difluoro-piperidin-1-yl)-8-(2-hydroxy-ethyl)-5,6,7,8-tetrahydro-pteridine-2-carbonitrile
4-Cyclopentylamino-5,6,7,8-tetrahydro-pteridine-2-carbonitrile
4-Isobutylamino-5,6,7,8-tetrahydro-pteridine-2-carbonitrile
8-(2-Amino-ethyl)-4-piperidin-1-yl-5,6,7,8-tetrahydro-pteridine-2-carbonitrile
8-(4-Chlorophenyl)-4-morpholin-4-yl-6-oxo-5,6,7,8-tetrahydropteridine-2-carbonitrile
   and pharmaceutically acceptable salts thereof.

The present invention further provides a process for the preparation of a compound of formula (I). These are detailed in scheme 1.

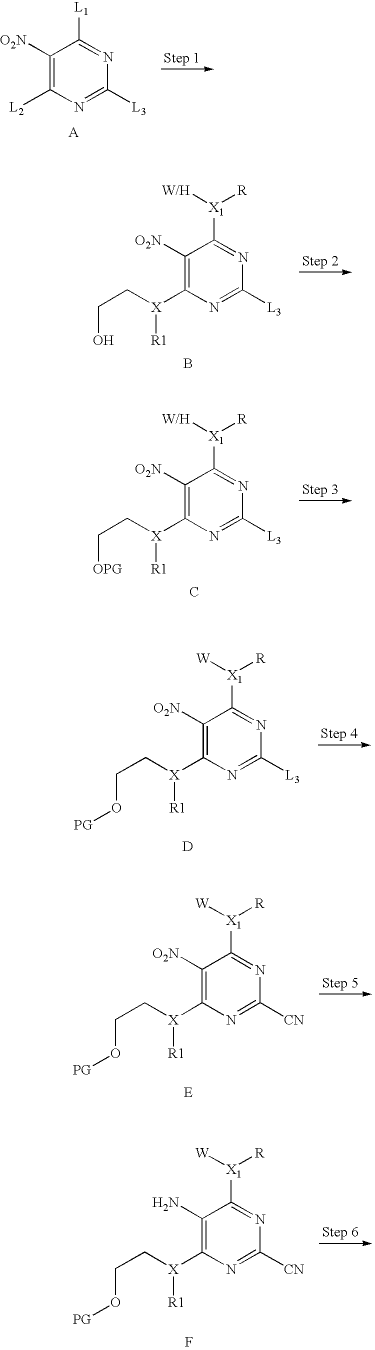

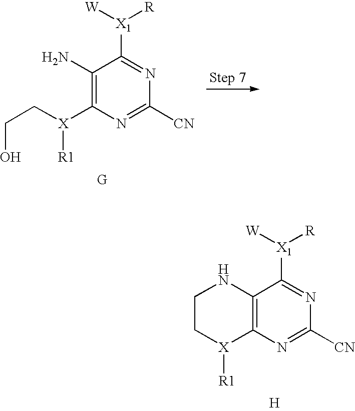

L1 and L2 may be displaced by X1R and XR1 respectively where R and R¹ are defined in formula (I) and L3 may be displaced by a cyanide salt. The sequence of displacement of L1, L2 and L3 may be varied.

### Step 1

Typically L1, L2 and L3 represent a leaving group (e.g. halide, sulphide, sulfoxide or sulphone group), preferably the sulphide is oxidised to a sulphoxide or sulphone group before displacement. An oxidising agent such as a peracid may be used, for example metachloroperbenzoic acid in dichloromethane at room temperature. Typically A is heated with amines either in the presence or absence of microwave irradiation. Solvents can be used if necessary.

### Step 2

The alcohol can be protected with a variety of protecting groups. These can be incorporated generally by reaction of the alcohol and the activated protecting group (i.e. trialkylsilyl chloride) using a base (organic/inorganic) in a suitable solvent (e.g. tetrahydrofuran, dichloromethane etc.).

### Step 3

If R1=H alkylation can be achieved using an alkylating agent usually in the presence of a base. Typical bases include sodium hydride, sodium ethoxide or potassium tert-butoxide in solvents such as tetrahydrofuran

### Step 4

W can be converted to the nitrile by reaction with a cyanide salt, usually in the presence of a solvent such as dimethylsulphoxide

### Step 5

The nitro group can be reduced under a variety of conditions. These include hydrogenation using a suitable catalyst (e.g. palladium on carbon) under a hydrogen atmosphere, transfer hydrogenation using cyclohexene or ammonium formate and suitable catalyst, or the use of metal mediated reductions such as tin II chloride, iron powder and suitable co reductant etc.

### Step 6

The protecting group can be removed in the presence of other functionality. For the silyl protecting group this can be achieved using acidic conditions, typically hydrogen chloride solution, or the use of fluoride ion (typically tetrabutyl ammonium fluoride solution in tetrahydrofuran)

### Step 7

Cyclisation can be affected by an iridium catalysed oxidative cyclisation in the presence of a base.

Compounds of the type highlighted can also be prepared as highlighted in scheme 2

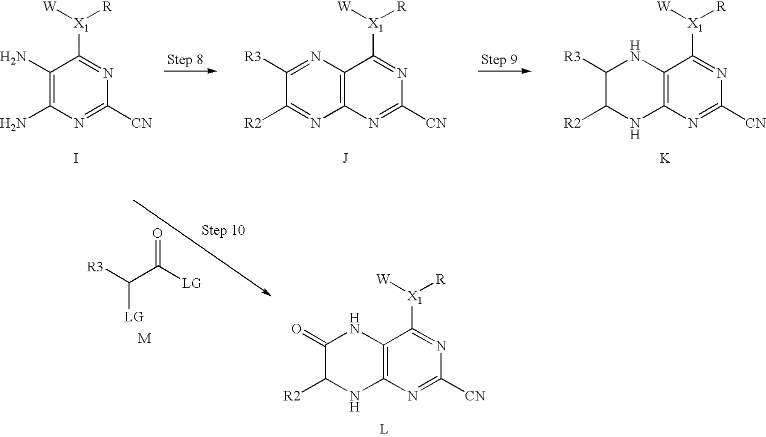

### Step 8

Condensation of a compound of type I with a requisite 1,2-dicarbonyl containing electrophile (aldehyde or ketone i.e. glyoxal) allows compounds of the type J to be formed.

### Step 9

J can be reduced with appropriate reducing agents (such a sodium borohydride) to furnish compounds of type K

### Step 10

Compounds of type I can also be reacted with compounds of type M under the presence of a base to afford the cyclised lactams L. Examples of compounds of type M include chloroacetylchloride.

According to a further feature of the invention there is provided a compound of the formula (I), or a pharmaceutically acceptable salt thereof, for use as a therapeutic agent.

According to a further feature of the present invention there is provided a method for producing inhibition of a cysteine protease in a warm blooded animal, such as man, in need of such treatment, which comprises administering to said animal an effective amount of a compound of the present invention, or a pharmaceutically acceptable salt thereof.

The invention also provides a compound of the formula (I), or a pharmaceutically acceptable salt thereof, for use as a medicament; and the use of a compound of the formula (I) of the present invention, or a pharmaceutically acceptable salt thereof, in the manufacture of a medicament for use in the inhibition of a cysteine protease in a warm blooded animal, such as man. In particular the compounds of the invention are useful in the treatment of inflammation and immune disorders such as, but not limited to, asthma, rheumatoid arthritis, COPD, multiple sclerosis, Crohn's disease, Alzheimers and pain, such as neuropathic pain. Preferably the compounds of the invention are used to treat pain, especially neuropathic pain.

In particular the invention provides the use of a compound of the formula (I) of the present invention, or a pharmaceutically acceptable salt thereof, in the manufacture of a medicament for use in the inhibition of Cathepsin K in a warm blooded animal, such as man. In order to use a compound of the formula (I) or a pharmaceutically acceptable salt thereof for the therapeutic treatment of mammals including humans, in particular in the inhibition of a cysteine protease, it is normally formulated in accordance with standard pharmaceutical practice as a pharmaceutical composition.

Therefore in another aspect the present invention provides a pharmaceutical composition which comprises a compound of the formula (I) or a pharmaceutically acceptable salt thereof and a pharmaceutically acceptable diluent or carrier.

The pharmaceutical compositions of this invention may be administered in standard manner for the disease condition that it is desired to treat, for example by oral, rectal or parenteral administration. For these purposes the compounds of this invention may be formulated by means known in the art into the form of, for example, tablets, capsules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 100 mg and 1 g of the compound of this invention.

In another aspect a pharmaceutical composition of the invention is one suitable for intravenous, subcutaneous or intramuscular injection.

Each patient may receive, for example, an intravenous, subcutaneous or intramuscular dose of 1 mgkg⁻¹ to 100 mgkg⁻¹ of the compound, preferably in the range of 5 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), or a pharmaceutically-acceptable salt thereof (hereafter compound X), for therapeutic or prophylactic use in humans:

(a)

| Tablet I | mg/tablet |
|---|---|
| Compound X. | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1. |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β cyclodextrin may be used to aid formulation.

### Note

The above formulations may be obtained by conventional procedures well known in the pharmaceutical art. The tablets (a)-(c) may be enteric coated by conventional means, for example to provide a coating of cellulose acetate phthalate.

The following examples illustrate the invention.

### EXAMPLE 1

### 8-(2-Benzyloxy-ethyl)-4-(cyclopentyl-methyl-amino)-5,6,7,8-tetrahydro-pteridine-2-carbonitrile

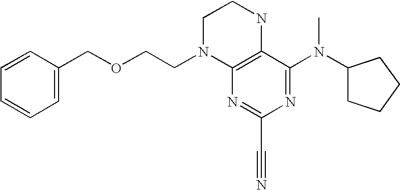

A solution of 5-Amino-4-[(2-benzyloxy-ethyl)-(2-hydroxy-ethyl)-amino]-6-(cyclopentyl-methyl-amino)-pyrimidine-2-carbonitrile (121 mgs, 0.295 mmol), pentamethylcyclopentadienyliridium(III) chloride dimer (12 mgs, 5 mol %) and potassium carbonate (4 mgs, 10 mol %) in trifluoromethylbenzene (3 ml) under a nitrogen atmosphere was heated at 180° C. under microwave irradiation for 40 mins (Machine/power etc). An additional quantity of pentamethylcyclopentadienyliridium(III) chloride dimer (12 mgs, 5 mol %) was added and heating/irradiation continued for another 40 mins. The solution was washed with aqueous sodium hydrogen carbonate and purified by reverse phase Hplc (add method) to afford 8-(2-Benzyloxy-ethyl)-4-(cyclopentyl-methyl-amino)-5,6,7,8-tetrahydro-pteridine-2-carbonitrile (4.5 mgs) as a colourless oil.

LCMS 710275 [MH]+392

1H NMR: (CDCl3) δ 7.2-7.4 (5H, m), 4.55 (2H, s), 4.2 (1H, m), 3.9 (2H, m), 3.75 (2H, m), 3.7 (2H, m), 3.45 (2H, m), 3.15 (3H, s), 1.8-2.0 (6H, m), 1.65 (2H, m)

The starting material, 5-Amino-4-[(2-benzyloxy-ethyl)-(2-hydroxy-ethyl)-amino]-6-(cyclopentyl-methyl-amino)-pyrimidine-2-carbonitrile, was prepared as described below:

### Step 1

A mixture of benzyloxyacetaldehyde (2.03 g, 13.5 mmol) and ethanolamine (743 ul, 12.1 mmol) in ethanol 40 ml was stirred at room temperature for 30 mins before cooling to 0° C. Sodium borohydride (616 mgs, 16.2 mmol) was added and the mixture stirred at 0° C. for 30 mins. The mixture was passed through an isolute flash SCX-2 cartridge and flushed with methanol. The product was eluted using 2M ammonia solution in methanol. The fractions were combined and concentrated to afford 2-(2-Benzyloxy-ethylamino)-ethanol (1.97 g) as a colourless oil which was used without further purification.

### Step 2

To a solution of 4,6-Dichloro-2-methylsulfanyl-5-nitro-pyrimidine (2.42 g, 10 mmol, WO9828300) and diisopropylethylamine (3.56 ml, 20 mmol) in ethanol (25 ml) stirred at 0° C. was added 2-(2-Benzyloxy-ethylamino)-ethanol (1.97 g, 10 mmol). After stirring for 10 min, cyclopentylamine (851 mg, 10 mmol) was added and the mixture heated at 120° C. using microwave irradiation for 10 min. The mixture was cooled and evaporated under reduced pressure to afford 2-[(2-Benzyloxy-ethyl)-(6-cyclopentylamino-2-methylsulfanyl-5-nitro-pyrimidin-4-yl)-amino]-ethanol (4.2 g) as a colourless oil which was used without further purification.

### Step 3

A solution of 2-[(2-Benzyloxy-ethyl)-(6-cyclopentylamino-2-methylsulfanyl-5-nitro-pyrimidin-4-yl)-amino]-ethanol (3.1 g, 6.9 mmol), tert-butyldimethylsilyl chloride (1.04 g, 6.9 mmol) and imidazole (563 mgs, 7.6 mmol) in dimethylformamide (15 ml) were stirred at room temperature for 1 hour. The mixture was diluted with ethyl acetate (150 ml) and washed with water (3×100 ml). The organic layer was dried (sodium sulphate), filtered and evaporated under reduced pressure. The residue was purified by column chromatography (cyclohexane to 2.5% ethyl acetate in cyclohexane) to afford N-(2-Benzyloxy-ethyl)-N-[2-(tert-butyl-dimethyl-silanyloxy)-ethyl]-N-cyclopentyl-2-methylsulfanyl-5-nitro-pyrimidine-4,6-diamine (2.14 g) as a colourless oil.

1H NMR: (CDCl3) δ 7.2-7.4 (5H, m), 4.55 (3H, m), 3.8 (2H, m), 3.75 (2H, m), 3.75 (4H, m), 3.6 (2H, m), 2.45 (3H, s), 2.0-2.1 (2H, m), 1.7-1.8 (2H, m), 1.6-1.7 (2H, m), 1.45-1.55 (2H, m), 1.4 (9H, s), 0.8 (6H, s)

### Step 4

To a solution of N-(2-Benzyloxy-ethyl)-N-[2-(tert-butyl-dimethyl-silanyloxy)-ethyl]-N′-cyclopentyl-2-methylsulfanyl-5-nitro-pyrimidine-4,6-diamine (450 mgs, 0.8 mmol) in tetrahydrofuran (5 ml) was added sodium hydride (35 mgs, 0.88 mmol). The solution was stirred at room temperature for 10 mins and methyl iodide (7.5 ul, 1.2 mmol) added. The mixture was stirred for 24 hrs with an additional quantity of sodium hydride (35 mgs) and methyl iodide (7.5 ul) added after 2 hrs. The mixture was portioned between ethyl acetate and water (100 ml each). The organic layer was separated, dried over sodium sulphate, filtered and evaporated to afford N-(2-Benzyloxy-ethyl)-N-[2-(tert-butyl-dimethyl-silanyloxy)-ethyl]-N′-cyclopentyl-N′-methyl-2-methylsulfanyl-5-nitro-pyrimidine-4,6-diamine (420 mgs) as a yellow oil which was used without further purification.

### Step 5

A solution of N-(2-Benzyloxy-ethyl)-N-[2-(tert-butyl-dimethyl-silanyloxy)-ethyl]-N′-cyclopentyl-N′-methyl-2-methylsulfanyl-5-nitro-pyrimidine-4,6-diamine (420, 0.73 mmol) and m-chloroperbenzoic acid (70%, 360 mgs, 1.46 mmol) in dichloromethane (20 mls) was stirred at room temperature for 10 mins. The reaction mixture was diluted with additional dichloromethane (30 mls) and washed with sodium thiosulphate, water, sodium hydrogen carbonate. The organic layer was dried, filtered and evaporated. The crude residue was dissolved in dimethyl sulphoxide and sodium cyanide (36 mgs, 0.73 mmol) added. The mixture was stirred at room temperature for 10 mins and partitioned between ethyl acetate and water. The organic layer was separated, dried over sodium sulphate, filtered and concentrated to afford 4-{(2-Benzyloxy-ethyl)-[2-(tert-butyl-dimethyl-silanyloxy)-ethyl]-amino}-6-(cyclopentyl-methyl-amino)-5-nitro-pyrimidine-2-carbonitrile (410 mgs) as a yellow oil which was used without further purification.

### Step 6

A mixture of 4-{(2-Benzyloxy-ethyl)-[2-(tert-butyl-dimethyl-silanyloxy)-ethyl]-amino}-6-(cyclopentyl-methyl-amino)-5-nitro-pyrimidine-2-carbonitrile (410, mgs), 10% Palladium on Carbon (130 mgs) in ethyl acetate (25 mls) was stirred under a hydrogen atmosphere for 6 hrs. The mixture was filtered and the solvent removed under reduced pressure to afford 5-Amino-4-{(2-benzyloxy-ethyl)-[2-(tert-butyl-dimethyl-silanyloxy)-ethyl]-amino}-6-(cyclopentyl-methyl-amino)-pyrimidine-2-carbonitrile (240 mgs) as a brown oil which was used without further purification

### Step 7

A solution of 5-Amino-4-{(2-benzyloxy-ethyl)-[2-(tert-butyl-dimethyl-silanyloxy)-ethyl]-amino}-6-(cyclopentyl-methyl-amino)-pyrimidine-2-carbonitrile (240 mgs) and 1M tetrabutylammonium fluoride (0.75 ml) in tetrahydrofuran (5 ml) was stirred at room temperature for 1 hr. Water (5 ml) was added and the mixture concentrated to dryness. The crude product was purified by column chromatography (50% ethyl acetate in cyclohexane) to afford 5-Amino-4-[(2-benzyloxy-ethyl)-(2-hydroxy-ethyl)-amino]-6-(cyclopentyl-methyl-amino)-pyrimidine-2-carbonitrile (178 mgs) as a colourless oil.

LCMS [MH]+411

1H NMR: (CDCl3) δ 7.2-7.4 (5H, m), 4.65 (1H, m), 4.55 (2H, s), 3.95 (2H, m), 3.75 (2H, m), 3.65 (2H, m), 3.5 (2H, m), 2.7 (3H, s), 1.8-1.85 (2H, m), 1.7 (2H, m), 1.5-1.65 (4H, m)

### EXAMPLE 2

### 8-(2-Benzyloxy-ethyl)-4-piperidin-1-yl-5,6,7,8-tetrahydro-pteridine-2-carbonitrile

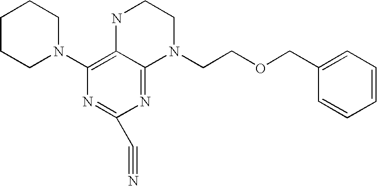

Prepared according to example 1 (stating materials synthesised using steps 2, 3, 5, 6 & 7 but substituting cyclopentylamine for piperidine)

LCMS [MH]+379

1H NMR: (CDCl3) δ 7.2-7.4 (5H, m), 4.5 (2H, s), 4.0 (2H, m), 3.75 (2H, m), 3.6 (2H, m), 3.65 (2H, m), 3.35 (2H, m), 3.05 (3H, s), 1.55-1.7 (6H, m)

### EXAMPLE 3

### 8-Cyclopentyl-4-morpholin-4-yl-5,6,7,8-tetrahydro-pteridine-2-carbonitrile

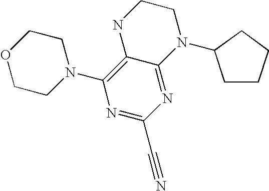

Prepared according to example 1 (stating materials synthesised using steps 1, 2, 3, 5, 6 & 7 but substituting cyclopentylamine for morpholine and benzyloxyacetaldehyde with cyclopentanone)

[MH]+315

1H NMR: (CDCl3) δ 5.15-5.2 (1H, m), 3.95-4.0 (1H, brs), 3.8 (4H, m), 3.45 (2H, m), 3.35 (2H, m), 3.1 (4H, m), 1.8-1.9 (2H, m), 1.6-1.8 (4H, m), 1.5 (2H, m)

### EXAMPLE 4

### 4-Morpholin-4-yl-8-phenyl-5,6,7,8-tetrahydro-pteridine-2-carbonitrile

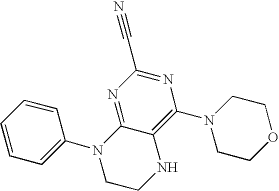

Prepared according to example 1 (stating materials synthesised using steps 2, 5& 6 but substituting cyclopentylamine for morpholine and 2-(2-Benzyloxy-ethylamino)-ethanol with N-phenylethanolamine)

LCMS [MH]+323

1H NMR: (CDCl3) δ 7.4 (2H, m), 7.3 (2H, m), 7.25 (1H, m), 3.95 (2H, m), 3.85 (4H, m), 3.55 (2H, m), 3.2 (4H, m)

### EXAMPLE 5

### 8-(2,2-Dimethyl-propyl)-4-morpholin-4-yl-5,6,7,8-tetrahydro-pteridine-2-carbonitrile

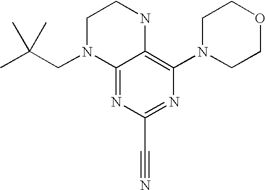

Prepared according to example 1 (stating materials synthesised using steps 1, 2, 3, 5, 6 & 7 but substituting cyclopentylamine for morpholine and benzyloxyacetaldehyde with trimethylacetaldehyde)

LCMS [MH]+317

1H NMR: (CDCl3) δ 3.85 (4H, m), 3.6 (2H, m), 3.45 (2H, s), 3.4 (2H, m), 3.15 (4H, m), 0.95 (9H, s)

### EXAMPLE 6

### 8-(2-Hydroxy-ethyl)-4-piperidin-1-yl-5,6,7,8-tetrahydro-pteridine-2-carbonitrile

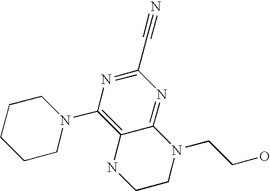

To a solution of 8-(2-Benzyloxy-ethyl)-4-piperidin-1-yl-5,6,7,8-tetrahydro-pteridine-2-carbonitrile (example 2, 160 mgs, 0.43 mmol) in dichloromethane stirred at −78° C. was added a 1M solution of boron trichloride in dichloromethane (1.5 ml). The reaction mixture was allowed to warm to room temperature over 1 hr and was quenched by additional of methanol. The mixture was concentrated to dryness and purified by column chromatography (33% ethyl acetate in cyclohexane) to afford 8-(2-Hydroxy-ethyl)-4-piperidin-1-yl-5,6,7,8-tetrahydro-pteridine-2-carbonitrile (27 mgs) as a red oil.

¹H NMR: (CDCl3) δ 4.0 (1H, brs), 3.85 (2H, m), 3.75 (2H, m), 3.65 (2H, m), 3.42 (2H, m), 3.05 (4H, m), 1.55-1.7 (6H, m)

### EXAMPLE 7

### 4-(4,4-Difluoro-piperidin-1-yl)-8-(2-hydroxy-ethyl)-5,6,7,8-tetrahydro-pteridine-2-carbonitrile

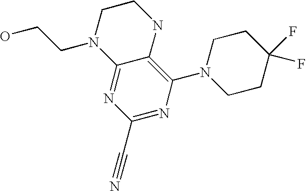

Prepared according to example 6 but substituting 8-(2-Benzyloxy-ethyl)-4-piperidin-1-yl-5,6,7,8-tetrahydro-pteridine-2-carbonitrile with 4-(4,4-Difluoro-piperidin-1-yl)-8-(2-benzyloxy-ethyl)-5,6,7,8-tetrahydro-pteridine-2-carbonitrile. 4-(4,4-Difluoro-piperidin-1-yl)-8-(2-benzyloxy-ethyl)-5,6,7,8-tetrahydro-pteridine-2-carbonitrile was prepared in an analogous manner to 8-(2-Benzyloxy-ethyl)-4-piperidin-1-yl-5,6,7,8-tetrahydro-pteridine-2-carbonitrile (example 2 but substituting piperidine for 4,4-difluoropiperidine.

LCMS [MH]+325

1H NMR: (CDCl3) δ 3.9 (2H, m), 3.75 (2H, m), 3.66 (2H, m), 3.45 (2H, m), 3.35 (4H, m), 2.3 (1H, brs), 2.0-2.25 (4H, m)

### EXAMPLE 8

### 4-Cyclopentylamino-5,6,7,8-tetrahydro-pteridine-2-carbonitrile

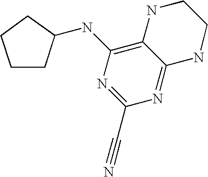

A mixture of 4,5-Diamino-6-cyclopentylamino-pyrimidine-2-carbonitrile (40 mgs, 0.18 mmol) glyoxal (40% solution, 40 ul) in ethanol was heated at 120° C. using microwave irradiation for 10 mins. The mixture was cooled to room temperature and sodium borohydride (15 mg, 2.2 mmol) added. The reaction was stirred at room temperature for 10 mins, the reaction mixture acidified with 1M HCl and concentrated under reduced pressure. The residue was purified using column chromatography (25-45% ethyl acetate in cyclohexane) to afford 4-Cyclopentylamino-5,6,7,8-tetrahydro-pteridine-2-carbonitrile (11 mgs) as a white solid.

LCMS [MH]+245

1H NMR: (CDCl3) δ 4.2-4.4 (2H, brs), 4.1 (1H, m), 3.2-3.6 (4H, m), 2.0-2.1 (2H, m), 1.55-1.7 (4H, m), 1.3-1.4 (2H, m)

The starting material, 4,5-Diamino-6-cyclopentylamino-pyrimidine-2-carbonitrile, was prepared as described below:

### Step 1

Cyclopentyl-(2,4-dimethoxy-benzyl)-amine was prepared according to example 1 step 1 but using cyclopentylamine and 2,4-dimethoxybenzaldehyde instead of ethanolamine and benzyloxyacetaldehyde and used without further purification.

### Step 2

N-Cyclopentyl-N,N′-bis-(2,4-dimethoxy-benzyl)-2-methylsulfanyl-5-nitro-pyrimidine-4,6-diamine was prepared according to example 1 step 2 but using Cyclopentyl-(2,4-dimethoxybenzyl)-amine and 2,4-dimethoxybenzylamine instead of 2-(2-Benzyloxy-ethylamino)ethanol and cyclopentylamine.

### Step 3

4-[Cyclopentyl-(2,4-dimethoxy-benzyl)-amino]-6-(2,4-dimethoxy-benzylamino)-5-nitro-pyrimidine-2-carbonitrile was prepared according to example 1 step 5 but replacing N-(2-Benzyloxy-ethyl)-N-[2-(tert-butyl-dimethyl-silanyloxy)-ethyl]-1-cyclopentyl-N′-methyl-2-methylsulfanyl-5-nitro-pyrimidine-4,6-diamine with N-Cyclopentyl-N,N′-bis-(2,4-dimethoxy-benzyl)-2-methylsulfanyl-5-nitro-pyrimidine-4,6-diamine and was used without further purification.

### Step 4

A mixture of 4-[Cyclopentyl-(2,4-dimethoxy-benzyl)-amino]-6-(2,4-dimethoxy-benzylamino)-5-nitro-pyrimidine-2-carbonitrile (1.13 g, 2.06 mmol) and trifluoroacetic acid (10 mls) in dichloroethane (10 mls) was stirred at 0° C. for 30 mins. The mixture was concentrated under reduced pressure and the residue purified by column chromatography (10% ethyl acetate in cyclohexane) to afford 4-Amino-6-cyclopentylamino-5-nitro-pyrimidine-2-carbonitrile (461 mgs) as a yellow solid.

LCMS [MH]+249

1H NMR: (CDCl3) δ 9.2 (1H, brs), 8.6 (1H, brs), 6.3 (1H, brs), 4.6 (1H, m), 2.1-2.2 (2H, m), 1.65-1.8 (4H, m), 1.5-1.6 (2H, m)

### Step 5

4,5-Diamino-6-cyclopentylamino-pyrimidine-2-carbonitrile was prepared according to example 1 step 6 but replacing 4-{(2-Benzyloxy-ethyl)-[2-(tert-butyl-dimethyl-silanyloxy)-ethyl]-amino}-6-(cyclopentyl-methyl-amino)-5-nitro-pyrimidine-2-carbonitrile with 4-Amino-6-cyclopentylamino-5-nitro-pyrimidine-2-carbonitrile. and used without further purification.

### EXAMPLE 9

### 4-Isobutylamino-5,6,7,8-tetrahydro-pteridine-2-carbonitrile

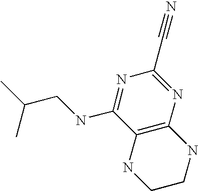

Prepared according to example 8 but replacing 4,5-Diamino-6-cyclopentylamino-pyrimidine-2-carbonitrile with 4,5-Diamino-6-isobutylamino-pyrimidine-2-carbonitrile.

LCMS [MH]+233

1H NMR: (CDCl3) δ 5.85 (1H, brs), 4.6 (1H, brs), 3.5 (2H, m), 3.35 (2H, m), 3.25 (2H, m), 2.6 (1H, brs), 1.8-1.9 (1H, m), 0.95 (6H, d)

### EXAMPLE 10

### 8-(2-Amino-ethyl)-4-piperidin-1-yl-5,6,7,8-tetrahydro-pteridine-2-carbonitrile

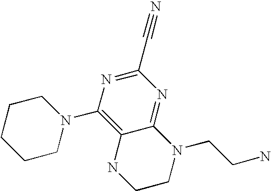

A solution of 8-[2-(1,3-Dioxo-1,3-dihydro-isoindol-2-yl)-ethyl]-4-piperidin-1-yl-5,6,7,8-tetrahydro-pteridine-2-carbonitrile (9 mgs, 0.0216 mmol) and hydrazine hydrate (20 mgs) in ethanol (5 ml) were stirred at 70° C. for 5 hrs. The mixture was purified by reverse phase Hplc to afford 8-(2-Amino-ethyl)-4-piperidin-1-yl-5,6,7,8-tetrahydro-pteridine-2-carbonitrile (4 mgs) as the trifluoroacetate salt.

LCMS [MH]+288

1H NMR: (CDCl3) δ 3.85 (2H, m), 3.75 (2H, m), 3.6 (2H, m), 3.45 (2H, m), 3.2 (2H, m), 3.05-3.1 (4H, m), 1.55-1.7 (6H, m)

8-[2-(1,3-Dioxo-1,3-dihydro-isoindol-2-yl)-ethyl]-4-piperidin-1-yl-5,6,7,8-tetrahydro-pteridine-2-carbonitrile was prepared as follows;

To a solution of 8-(2-Hydroxy-ethyl)-4-piperidin-1-yl-5,6,7,8-tetrahydro-pteridine-2-carbonitrile (28 mgs, 0.1 mmol), triphenylphosphine (40 mgs, 0.15 mmol) and phthalimide (20 mgs, 0.136 mmol) in tetrahydrofuran was added diethylazodicarboxylate (25 mgs, 0.17 mmol) and the mixture stirred at room temperature for 20 hrs. The mixture was diluted with ethyl acetate and washed with saturated sodium hydrogen carbonate solution. The organic layer was passed down an isolute flash SCX-2 cartridge and flushed with methanol. The product was eluted using 2M ammonia solution in methanol. The basic fractions were combined and concentrated under reduced pressure to afford 8-[2-(1,3-Dioxo-1,3-dihydro-isoindol-2-yl)-ethyl]-4-piperidin-1-yl-5,6,7,8-tetrahydro-pteridine-2-carbonitrile (17 mgs) as an orange solid which was used without further purification.

### EXAMPLE 11

### 8-(4-Chlorophenyl)-4-morpholin-4-yl-6-oxo-5,6,7,8-tetrahydropteridine-2-carbonitrile

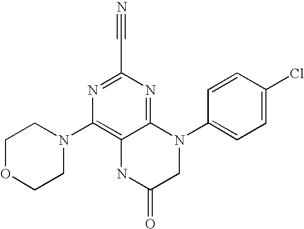

A solution of 5-Amino-4-(4-chloro-phenylamino)-6-morpholin-4-yl-pyrimidine-2-carbonitrile (0.2 g) (prepared according to WO2004000819), triethylamine (0.33 ml) and chloroacetyl chloride (50 δl) in dichloromethane (10 ml) was stirred at room temperature for 1 h, then a further 50 δl of chloroacetyl chloride was added. After 3 h, triethylamine (0.3 ml) then a further portion of chloroacetyl chloride (0.2 ml) was added and the mixture stirred at room temperature for 16 h. The solvent was evaporated and the residue purified by chromatography on silica eluting with 50% ethyl acetate/isohexane. The residue was dissolved in acetonitrile (10 ml) then triethylamine (0.5 ml) added and the mixture heated at 60° C. for 3 h. The solvent was evaporated under reduced pressure and the residue purified by chromatography on silica eluting with 3% methanol/dichloromethane then by RPHPLC.

[MH]+371

1H NMR: (DMSO-d6) δ 10.58 (1H, s), 7.50 (2H, d), 7.41 (2H, d), 4.39 (2H, s), 3.72-3.74 (4H, m), 3.29-3.32 (4H, m)

### Assay for Identification of Cathepsin K Inhibitors

QFRET Technology (Quenched Fluorescent Resonance Energy Transfer) was used to measure the inhibition by test compounds of cathepsin K-mediated cleavage of the synthetic peptide Z-Phe-Arg-AMC. Compounds were screened at twelve concentrations (3.5×10−8-10 uM), on two separate occasions and the mean pIC50 values reported.

0.5 nM [final] rhuman cathepsin K in phosphate buffer was added to a 384-well black microtitre plate containing investigative compounds. The enzyme and compound were pre-incubated at room temperature for 30 minutes before the addition of 50 mM [final] Z-Phe-Arg-AMC synthetic substrate in phosphate buffer. The plates were covered and incubated for 1 h at room temperature, protected from light. Following the incubation the reaction was stopped with 7.5% [final] acetic acid. Relative fluorescence was measured using the Ultra plate reader at a wavelength of 360 nm excitation and 425 nm emission.

Data was corrected for background fluorescence (minimum controls without enzyme). This data was used to plot inhibition curves and calculate pIC50 values by non-linear regression using a variable slope, offset=zero model in Origin 7.5 analysis package. Reproducibility of data was assessed using a quality control statistical analysis package whereby internal variability of the assayed indicated a repeat testing (n=3) if pIC50 SD was >0.345.

### REFERENCES

Dieppe P A 2005 Arthritis Rheum. 52(11):3536-41. Assessing bone loss on radiographs of the knee in osteoarthritis: a cross-sectional study.
Dodds R A 1999 Arthritis Rheum. 42(8):1588-93. Expression of cathepsin K messenger RNA in giant cells and their precursors in human osteoarthritic synovial tissues.
Felson D T 2001 Ann Intern Med. 134(7):541-9. The association of bone marrow lesions with pain in knee osteoarthritis.
Gowen M 1999 J Bone Miner Res. 14(10):1654-63. Cathepsin K knockout mice develop osteopetrosis due to a deficit in matrix degradation but not demineralization.
Kafienah W 1998 Biochem J. 1; 331 (Pt 3):727-32. Human cathepsin K cleaves native type I and II collagens at the N-terminal end of the triple helix.
Karvonen R L 1998 J Rheumatol. 25(11):2187-94. Periarticular osteoporosis in osteoarthritis of the knee.
Konttinen Y T 2002 Arthritis Rheum. 2002; 46(4):953-60. Acidic cysteine endoproteinase cathepsin K in the degeneration of the superficial articular hyaline cartilage in osteoarthritis.

Lindeman J H 2004 Am J Pathol. 165(2):593-600. Cathepsin K is the principal protease in giant cell tumor of bone.
Messent E A 2005 Osteoarthritis Cartilage. 13(1):39-47. Cancellous bone differences between knees with early, definite and advanced joint space loss; a comparative quantitative macroradiographic study.
Morko J 2005 Arthritis Rheum. 52(12):3713-7. Spontaneous development of synovitis and cartilage degeneration in transgenic mice overexpressing cathepsin K.
Radin E L, Rose R M. 1986 Clin Orthop Relat Res. (213):34-40. Role of subchondral bone in the initiation and progression of cartilage damage.
Selinger C I 2005 J Cell Biochem. 96(5):996-1002. Optimized transfection of diced siRNA into mature primary human osteoclasts: inhibition of cathepsin K mediated bone resorption by siRNA.
Shibakawa A 2005 Osteoarthritis Cartilage. 13(8):679-87. The role of subchondral bone resorption pits in osteoarthritis: MMP production by cells derived from bone marrow.
Skoumal M 2005 Arthritis Res Ther. 7(1):R65-70. Serum cathepsin K levels of patients with longstanding rheumatoid arthritis: correlation with radiological destruction.
Szebenyi B 2006 Arthritis Rheum. 54(1):230-5. Associations between pain, function, and radiographic features in osteoarthritis of the knee.
Tezuka K 1994 J Biol Chem.; 269(2):1106-9. Molecular cloning of a possible cysteine proteinase predominantly expressed in osteoclasts.

1. A compound of formula (I): 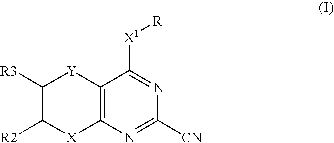
in which:
X is NR¹ or O;
Y is O or NR⁴;
X¹ is a bond, NH or Nalkyl,
R is a 4, 5, 6 or 7-membered saturated monocyclic or bicyclic ring optionally containing one or more O, S(O)n or N atoms which can be optionally substituted by alkyl, C₃₋₆ cycloalkyl, or a spirocyclic group comprising 3-5 membered rings or (CH₂)nX where X is amino, hydroxy, OR⁴, cyano, trifluoromethyl, carboxy, CONR⁵R⁶, SO₂NR⁵R⁶, SO₂R⁴, NR⁴SO₂R⁴, NR⁴COR⁴, C₁₋₆ alkyl, C₁₋₆ alkoxy, SR⁴ or NR⁵R⁶ where R⁴ is hydrogen, C₁₋₆ alkyl or C₃₋₆ cycloalkyl, R⁵ and R⁶ are independently hydrogen, C₁₋₆ alkyl, or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶;
or R is a group —(CH₂)nY(CH₂)pR⁷ where n and p are independently 0, 1 or 2 and Y is a bond, O, S(O)n or NR⁸ where R⁸ is hydrogen, C₁₋₆ alkyl or C₃₋₆ cycloalkyl;
R¹ is hydrogen, C₁₋₆ alkyl or C₃₋₇ cycloalkyl, both of which can be optionally substituted by alkyl (including branching), cycloalkyl, or (CH2)nX where X=amino, hydroxy, OR4, cyano, trifluoromethyl, carboxy, CONR⁵R⁶, SO₂NR⁵R⁶, SO₂R⁴, NR⁴SO₂R⁴, NR⁴COR⁴, C₁₋₆ alkyl, C₁₋₆ alkoxy, SR⁴ or NR⁵R⁶; or
R¹ is a group —(CH₂)nY(CH₂)pR⁷ where n and p are independently 0, 1 or 2 and Y is a bond, O, S(O)n or NR⁸ where R⁸ is hydrogen, C₁₋₆ alkyl or C₃₋₆ cycloalkyl;
R² is hydrogen or C₁₋₆ alkyl;
R³ is hydrogen or C₁₋₆ alkyl;
R⁴ is hydrogen, C₁₋₆ alkyl or C₃₋₆ cycloalkyl, and
R⁵ and R⁶ are independently hydrogen, C₁₋₆ alkyl;
R⁷ is a 3- to 7-membered saturated ring optionally containing one or more O, S or N atoms (sulphur may be in the form S(O)n), or an aryl or a heteroaryl group containing one to four heteroatoms selected from O, S or N, the saturated ring, aryl and heteroaryl groups all being optionally substituted by halogen, amino, hydroxy, cyano, nitro, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶; or R⁷ is hydrogen, amino, hydroxy, OR⁴, cyano, trifluoromethyl, carboxy, CONR⁵R⁶, SO₂NR⁵R⁶, SO₂R⁴, NHSO₂R⁴, NHCOR⁴, C₁₋₆ alkyl, C₁₋₆ alkoxy, SR⁴ or NR⁵R⁶,
and pharmaceutically acceptable salts or solvates thereof.

2. A compound according to claim 1 in which X is NR¹.

3. A compound according to claim 1 in which Y is NH or O.

4. A compound according to claim 1 in which X¹ is bond, NH or NMe.

5. A compound according to claim 1 in which R is a 5- or 6-membered saturated ring containing one or more O, S or N atoms.

6. A compound according to claim 1, in which R¹ hydrogen, C₁₋₆ alkyl substituted by hydroxyl or amino, C₃₋₆ cycloalkyl, phenyl or R¹ is a group —(CH₂)nO(CH₂)pR⁷ where n is 2 and p is 1 and R⁷ is phenyl.

7. A compound according to claim 1, in which R² and R³ are both hydrogen

8. A compound of formula (I) selected from:
8-(2-Benzyloxy-ethyl)-4-(cyclopentyl-methyl-amino)-5,6,7,8-tetrahydro-pteridine-2-carbonitrile
8-(2-Benzyloxy-ethyl)-4-piperidin-1-yl-5,6,7,8-tetrahydro-pteridine-2-carbonitrile
8-Cyclopentyl-4-morpholin-4-yl-5,6,7,8-tetrahydro-pteridine-2-carbonitrile
4-Morpholin-4-yl-8-phenyl-5,6,7,8-tetrahydro-pteridine-2-carbonitrile
8-(2,2-Dimethyl-propyl)-4-morpholin-4-yl-5,6,7,8-tetrahydro-pteridine-2-carbonitrile
8-(2-Hydroxy-ethyl)-4-piperidin-1-yl-5,6,7,8-tetrahydro-pteridine-2-carbonitrile
4-(4,4-Difluoro-piperidin-1-yl)-8-(2-hydroxy-ethyl)-5,6,7,8-tetrahydro-pteridine-2-carbonitrile
4-Cyclopentylamino-5,6,7,8-tetrahydro-pteridine-2-carbonitrile
4-Isobutylamino-5,6,7,8-tetrahydro-pteridine-2-carbonitrile
8-(2-Amino-ethyl)-4-piperidin-1-yl-5,6,7,8-tetrahydro-pteridine-2-carbonitrile
8-(4-Chlorophenyl)-4-morpholin-4-yl-6-oxo-5,6,7,8-tetrahydropteridine-2-carbonitrile
and pharmaceutically acceptable salts thereof.

9. (canceled)

10. A method of treating rheumatoid arthritis, osteoarthritis, Paget's disease, osteoporosis, invasive cancer, metastatic cancer or osteolytic bone cancer comprising administrating an effect amount of the compound of formula (I) as defined in claim 1 to a patient in need thereof.

11. A method of treating rheumatoid arthritis or osteoarthritis comprising administrating an effect amount of the compound of formula (I) as defined in claim 1 to a patient in need thereof.

12. A pharmaceutical composition which comprises a compound of the formula (I) as defined in claim 1 or a pharmaceutically acceptable salt thereof and a pharmaceutically acceptable diluent or carrier.

13. A method for producing inhibition of a cysteine protease in a mammal, such as man, in need of such treatment, which comprises administering to said mammal an effective amount of a compound as defined in claim 1, or a pharmaceutically acceptable salt thereof.

14. (canceled)

15. A method of treating rheumatoid arthritis, osteoarthritis, Paget's disease, osteoporosis, invasive cancer, metastatic cancer or osteolytic bone cancer comprising administrating an effect amount of the compound of formula (I) as defined in claim 8 to a patient in need thereof.

16. A method of treating rheumatoid arthritis or osteoarthritis comprising administrating an effect amount of the compound of formula (I) as defined in claim 8 to a patient in need thereof.

17. A pharmaceutical composition which comprises a compound of the formula (I) as defined in claim 8 or a pharmaceutically acceptable salt thereof and a pharmaceutically acceptable diluent or carrier.

18. A method for producing inhibition of a cysteine protease in a mammal, such as man, in need of such treatment, which comprises administering to said mammal an effective amount of a compound as defined in claim 8, or a pharmaceutically acceptable salt thereof.
